(19) United States
(12) Patent Application Publication
(10) Pub. No.: US 2016/0287552 A1
(43) Pub. Date: Oct. 6, 2016

(54) DRUG DELIVERY SYSTEM COMPRISING GELATINE NANO-PARTICLES FOR SLOWLY RELEASING HARDLY-WATER SOLUBLE SUBSTANCES AND ITS PREPARATION METHOD

(71) Applicant: INDUSTRY ACADEMIC COOPERATION FOUNDATION, DAEGU UNIVERSITY, Gyeongsangbuk-do (KR)

(72) Inventors: Kwang-Hee LIM, Daegu (KR); Eun-Ju LEE, Daegu (KR)

(73) Assignee: INDUSTRY ACADEMIC COOPERATION FOUNDATION, DAEGU UNIVERSITY, Gyeongsangbuk-do (KR)

(21) Appl. No.: 15/015,668

(22) Filed: Feb. 4, 2016

(30) Foreign Application Priority Data
Mar. 31, 2015 (KR) 10-2015-0045288

(51) Int. Cl. A61K 31/337; A61K 9/16; A61K 9/113

(52) U.S. Cl. CPC A61K 31/337; A61K 9/113; A61K 9/1682; A61K 9/1658; A61K 9/1617

(57) ABSTRACT

The present invention is about preparing gelatin nanoparticles having a size of about 200 nm are supported or not supported with a hardly-water soluble drug without a homogenizer by constructing O/W/O or W/O systems, thereby relatively prolonging the circulation time within the human body as compared to a water-repellent particle because it is free from the immune system, and enhancing EPR (Enhanced permeability and retention) effects. In this case, the hardly-water soluble drug includes hardly soluble anticancer agents such as paclitaxel, coenzyme Q10, ursodexoychlic acid, ilaprazole or imatinib mesylate. Furthermore, the O/W/O or W/O systems are nonpolar phase/polar phase/nonvolatile nonpolar phase and polar phase/nonvolatile nonpolar phase systems, respectively. More specifically, the O/W/O or W/O systems presents a hardly soluble drug/gelatin nanoparticle/fatty acid and gelatin nanoparticle/fatty acid systems, respectively.

### TECHNICAL FIELD

The present invention relates to a drug delivery system comprising gelatin nanoparticles which supports and slowly releases one or more of hardly-water soluble substances including paclitaxel, coenzyme Q10, ursodeoxycholic acid, ilaprazole or imatinib mesylate, and a method for preparing the same.

### BACKGROUND OF ART

Gelatin has a relatively low antigenicity and is mostly used in parenteral formulations. Also, gelatin consists of a protein structure having several kinds of functional groups and thus it can change its structure in multiple ways through the coupling of a targeting ligand, a crosslinking agent, a barrier material or the like and is used as a stabilizer in vaccines. Moreover, the extravascular medication was approved by FDA. In addition, since the hydrophilicity of gelatin can be protected from the immune system, gelatin nanoparticles can prolong the circulation time of gelatin within the human body. Meanwhile, for the drug to be delivered from the outside to the inside of the human body, the drug is dissolved, immobilized, encapsulated or absorbed in a nanoparticle matrix which is a drug carrier. Rejman et al. have reported that the size of the nanoparticles can have a significant impact on the absorption by cells and, in comparison with 50 nm particles, 200 nm particles have 3-4 times lower absorption and 200-500 nm particles have 8-10 times lower absortion, whereas in the case of the particles of greater than 1 m, the cellular absorption was not observed (Biochem. J. Immediate Publication, BJ2001253, 2003). Thus, the nanoparticles have several significant advantages including higher intracellular absorption as compared to microparticles.

In order to prepare gelatin nanoparticles, the emulsion/solvent evaporation method, reverse phase preparation method, coacervation/desolvation method and the like were used until recently, but there were problems in terms of the stability, the use of organic solvents and surfactants, and the difficulty of purification resulting therefrom. The coacervation/desolvation method refers to a method for producing a polymer-rich dense phase (coacervate) by performing the liquid-liquid phase separation through dehydrogenation by the process of adding a foreign material to a hydrophilic colloid bulk solution or changing the temperature. In an example of this method, Kaul and Amiji have recently reported that gelatine nanoparticles of 200-500 nm are prepared by adding ethanol as a gelatin bulk solution and controlling the gelatin precipitation while continuously stirring the mixture (Pharm. Research 19 (7), 2002). On the other hand, Fessi, H. C. et al. have prepared polymer nanoparticles of less than 500 nm by dissolving a polymer such as PLGA or PLA in a solvent and adding it to a non-solvent, using the nano-precipitation method (U.S. Pat. No. 593,522, 1990].

Paclitaxel is an anticancer substance that exist in nature and a drug made of diterpenoid derivatives extracted from periderm of taceae (Taxus brevifolia Nutt.). Paclitaxel is known to have efficacies against a variety of cancers such as lung cancers, breast cancers, etc. Paclitaxel basically as an alkaloid structure consisting of a taxane ring and an ester side chain, and exhibits poor solubility. Paclitaxel is an anticancer substance and is known to have efficacies against a variety of cancers such as lung cancer, breast cancer, etc. In order to solve the poor solubility of paclitaxel, conventionally it has been used by dissolving in ethanol, but has recently been used as an injection by binding to albumin in order to increase the delivery efficiency. In addition, a method of using a solvent called Cremorphor EL which is a mixture of polyoxyethylated castor oil and absolute ethanol has been known. However, clinically, when this solvent is administered in an excessive amount, it has been reported that it gives rise to side effects which cause cardiotoxicity and hypersensitivity reactions, and there is a need to urgently develop a method capable of improving a bioavailability by stably solubilizing paclitaxel. Thus, methods for solubilizing hardly-water soluble paclitaxel can be classified into four as follows.

Firstly, a hardly soluble paclitaxel is conjugagted with an amino acid of water-soluble polymer such as poly(L-glutamic acid). This method prepares a direct water-soluble macromolecule-conjugated paclitaxel. This shows an improved permeability against cancer vascular system and is accumulated in cancer tissues. However, the conjugated water-soluble paclitaxel has been reported to decrease toxicity on cancer cells in-vitro. Xyotax was studied and developed by Cell Therapeutics, but currently Novatis has acquisited it in 2006 and has the rights of development and sale (2006). The product name is Paclitaxel poliglumex (Xyotax; CT-2103; poly (L-glutamic acid)-paclitaxel conjugate; PPX), which is now during clinical trials for cancer of the head and neck.

Secondly, a hardly soluble paclitaxel is dissolved using a surfactant such as Cremophor EL or liposome as described above. In the case of excipients such as Cremophor EL, the dosage is limited due to side effects such as toxicity. In the case of liposome, there is a problem that it is physically instable and the amount of supported paclitaxel to be delivered is too small. However, paclitaxel available from Oasmia Pharmaceutical in Sweden, Paclical which is a nanoparticle formulation using retinoid-based excipient XR-17, is reported to have less side effects, and in the United States it has been designated as a rare drug for ovarian cancer in 2009. Further, Genexol-PM using micelles available from Samyang Genex can be mentioned.

Thirdly, a hardly soluble paclitaxel is prepared into a fine particle which can easily absorb based on microemulsion technology. This has been developed as a drug delivery system of oral paclitaxel by Hanmi Pharm. Co., Ltd.

Fourthly, a hardly soluble paclitaxel is supported in a water-soluble polymer such as gelatin. Ze Lu, et al. has reported that gelatin nanoparticles adsorbed with paclitaxel is prepared using a two step desolvation method (Clin. Cancer Res. 10: 7677-7684 (2004)). In the drug release experiment, paclitaxel adsorbed on a hydrophobic amino acid of gelatin nanoparticles was rapidly released within 5-6 hours, and thus slow release of the drug delivery has not been achieved. Such rapid release of paclitaxel can be occurred because paclitaxel is absorbed on the outer surface rather than the inside of gelatin nanoparticles in the preparation of gelatin nanoparticles using the two step desolvation method. Further, in order to enhance EPR (enhanced permeability and retention) effects on cancer cells of gelatin nanoparticles supported with paclitaxel, the nanoparticles having a size of about 200 nm is preferred, but the size of paclitaxel-supported gelatin nanoparticles manufactured by using the two step desolvation method is 600-900 nm, which is too large to expect EPR effects.

Meanwhile, by combining the first method with the fourth method, a hardly soluble paclitaxel and a water-soluble paclitaxel which is a conjugate of poly(L-glutamic acid) may be supported on a water-soluble polymer such as gelatin. In this case, the molecular weight of the water-soluble paclitaxel conjugated with the polymer is too large and thus the size of the gelatin particle supporting it may become much larger than the size of the gelatin particle supporting a monomer drug.

Thus, there is a need to develop a new technology to overcome the disadvantages of the method of solubilizing paclitaxel. Furthermore, this new technology can be applied even to a hardly-water soluble drug such as paclitaxel as well as coenzyme Q10, ursodexoychlic acid, ilaprazole or imatinib mesylate.

### DISCLOSURE OF INVENTION

### Technical Problem

The object of the present invention is to prepare gelatin nanoparticles having a size of about 200 nm supported or not supported with a hardly-water soluble drug without a homogenizer by constructing O/W/O or W/O systems, thereby relatively prolonging the circulation time within the human body as compared to a water-repellent particle because it is free from the immune system, and enhancing EPR (Enhanced permeability and retention) effects. In this case, the hardly-water soluble drug includes hardly soluble anticancer agents such as paclitaxel, coenzyme Q10, ursodexoychlic acid, ilaprazole or imatinib mesylate. Furthermore, the O/W/O or W/O systems refer to nonpolar phase/polar phase/nonvolatile nonpolar phase and polar phase/nonvolatile nonpolar phase systems, respectively. More specifically, the O/W/O or W/O systems refer to a hardly soluble drug/gelatin nanoparticle/fatty acid and gelatin nanoparticle/fatty acid systems, respectively.

### Technical Solution

According to the present invention, in order to make a hardly-water soluble drug into a fine particle which is soluble and easily absorbable, the O/W/O or W/O systems are constructed similarly to a nanoemulsion (or nanosuspension) method. Here, the O/W/O and W/O systems refer to nonpolar phase/polar phase/nonvolatile nonpolar phase and polar phase/nonvolatile nonpolar phase, respectively. More specifically, the O/W/O and W/O systems correspond to hardly soluble drug/gelatin nanoparticle/fatty acid and gelatin nanoparticle/fatty acid, respectively. In addition, the hardly-water soluble drug includes hardly soluble anticancer drugs such as paclitaxel, coenzyme Q10, ursodexoychlic acid, ilaprazole or imatinib mesylate.

Fatty acid may include oleic acid, linoleic acid or the like. Among them, in consideration of oral administration, linoleic acid which is converted into a conjugated linoleic acid capable of flowing at room temperature, inhibiting the proliferation of cancer cells by bifidobacterium strains in digestive organs of the human body and having anticancer effects is preferred. There is a report that the conjugated linoleic acid can penetrate a blood-brain barrier (BBB) which does not enable the drug to penetrate into a central nerve system [Fa et al., Biochim. Biophys Acta, 1736 (1), 61, 2005]. The anticancer agents for the treatment of many types of brain tumors that are injected by intravenous injection can not reach the brain tissue due to such blood-brain barrier (BBB) and thus shows a low therapeutic index in the brain cancers. Further, linoleic acid has beneficial properties to the skin and thus is often used in the cosmetic industry. When applied to the skin, linoleic acid has an anti-inflammatory effect, an acne-decreasing effect and a moisturizing effect.

Differently from conventional O/W/O or W/O emulsion systems, the present invention has been designed so that, without using a homogenizer, a solvent of a polar phase (W) gelatin solution is diffused in an oil phase (O) fatty acid and gelated while a gelatin particle is finely lowered to a nano-size. The solvent of the gelatin solution used in the present invention is a polar solvent except water and includes DMSO and the like.

In addition, in the present invention, a hardly soluble drug is supported in the inside of gelatin nanoparticles rather than the surface of gelatin nanoparticles as a drug carrier, thereby enhancing the slow release of supported drugs. Further, a mixture of hardly soluble drug-supported gelatin nanoparticles, fatty acids and gelatin-dissolving solvents is not purified or separated, and the mixture is used without any change or emulsified and then applied to an oral formulation for the treatment of brain cancers or gastrointestinal cancers or a skin-cancer target as a transdermal absorption anticancer agents against skin cancers or the like.

### Advantageous Effects

According to the present invention, the hardly-water soluble drug-supported gelatin nanoparticles of about 200 nm is prepared as a drug carrier from the gelatin solution to which the hardly-water soluble drug is added and thus, the circulation time of the drug carrier in the inside of the human body is relatively prolonged as compared to a water-repellent drug. Also, the present invention leads to several remarkable advantages including higher absorption in cells or EPR effects through size-reduction than the gelatin nanoparticles supported with paclitaxel of 600-900 nm prepared by two step desolvation method. Furthermore, the hardly soluble drug is not absorbed on the outer surface of gelatin nanoparticles as in the gelatin nanoparticles prepared by two step desolvation method, but the supported hardly soluble drug is placed inside the gelatin nanoparticles, thereby the supported hardly soluble drug has effects of enhancing a slow release as compared with the gelatin nanoparticles prepared by the two step desolvation method. In addition, the mixture of the hardly soluble drug-supported gelatin nanoparticles and fatty acid is not purified or separated, and the mixture is used without any change or emulsified and then applied to an oral formulation for the treatment of brain cancers or gastrointestinal cancers or a skin-cancer target as a transdermal absorption anticancer agents against skin cancers or the like.

### BRIEF DESCRIPTION OF THE DRAWING

FIG. 1 is a graph showing a change over time in the paclitaxel absorbance transition at 230 nm of the paclitaxel-supported gelatin nanoparticle samples prepared in accordance with Example 2 of the present invention.

FIG. 2 is a graph showing a change over time in the linoleic acid+paclitaxel absorbance transition at 205 nm of the paclitaxel-supported gelatin nanoparticle samples prepared in accordance with Example 2 of the present invention.

### BEST MODE FOR CARRYING OUT THE INVENTION

According to the present invention, in order to make a hardly-water soluble drug into a fine particle which is soluble and easily absorbable, the O/W/O or W/O systems are constructed similarly to a nanoemulsion (or nanosuspension) method without a homogenizer. Here, the O/W/O and W/O systems correspond to hardly soluble drug/gelatin nanoparticle/fatty acid and gelatin nanoparticle/fatty acid, respectively.

In order to prepare gelatin nanoparticles according to the present invention, gelatin is dissolved at 40-60° C. using a polar phase (W) solvent as a solvent, to prepare solutions to which a hardly soluble drug is added or not added. The hardly soluble drug includes one or two selected from the group consisting of paclitaxel, coenzyme Q10, ursodexoychlic acid, ilaprazole or imatinib mesylate. The polar phase (W) solvent includes DMSO. To the mixture of two or more of fatty acids was added dropwise or continuously added an aqueous solution of gelatin to which the hardly soluble drug was added or not added, to prepare the gelatin nanoparticles of about 200 nm. To the fatty acids, a surfactant can be added, and the surfactant includes sorbitan-based surfactants such as sorbitan monoisostearate, sorbitan monooleate, sorbitan sesquioleate or sorbitan trioleate. When the surfactant is added to the fatty acid and the hardly soluble drug is added to a gelatin solution, nanoemulsions (or nanosuspensions) from the O/W/O system are produced. Herein, the O/W/O system corresponds to hardly soluble drug/gelatin nanoparticle/fatty acid. Herein, since the gelatin nanoparticles are free from the immune system, the circulation time of the gelatin nanoparticles in the inside of the human body is relatively prolonged as compared to a water-repellent drug.

Fatty acid includes the same type of fatty acid such as oleic acid or linoleic acid or a mixture of several kinds of fatty acids. Among them, in consideration of oral administration, linoleic acid which is converted into conjugated linoleic acid capable of flowing at room temperature, inhibiting the proliferation of cancer cells by bifidobacterium strains in digestive organs of the human body and having anticancer effects is preferred. Further, linoleic acid has beneficial properties to the skin and thus is often used in the cosmetic industry. When applied to the skin, linoleic acid has an anti-inflammatory effect, an acne-decreasing effect and a moisturizing effect.

Differently from conventional O/W/O or W/O emulsion systems, the present invention has been designed so that, without using a homogenizer, a solvent of a polar phase (W) gelatin solution is diffused in an oil phase (O) fatty acid and gelated while a gelatin particle is finely lowered to a nano-size. The solvent of gelatin solution used in the present invention is a polar solvent except water and includes DMSO and the like.

Gelatin is an amphipathic material having a complex molecular structure, and contains both hydrophilic amino acids such as glycine, proline and hydroxyproline and water-repellent amino acid such as tryptophan, tyrosine, alanine, luecine and isoluecine. Therefore, the resulting gelatin nanoparticles can be surrounded by the hydrophilic portion of the surfactant on fatty acid as the amphipathic to improve the stability of the gelatin nanoparticles, and the hydrophilic side chain of the surrounded gelatin is in contact with the hydrophilic portion of the surfactant. In this case, the hardly soluble drug supported with the gelatin nanoparticles is in contact with the water-repellent side chain of gelatin in the inside of the gelatin nanoparticles. The hardly soluble drug supported during release of the drug can be slowly released in the outside of the gelatin nanoparticles from the inside of the gelatin nanoparticles. Accordingly, the present invention can enhance a slow release of the supported drug by placing the hardly-water soluble drug in the inside of the nanoparticle rather than the outer surface of the gelatin nanoparticles.

The step of injecting the surfactant is performed by adding a surfactant in an amount of 1 to 2 w/v % with respect to the volume of a non-solvent or 30 to 35 times of the total weight of the gelatin to fatty acid prior to the production of gelatin nanoparticles, or by injecting the surfactant to fatty acid within one hour after the production of gelatin nanoparticles. Even when not using a surfactant, gelatin nanoparticles may be produced and there is a stability that the nanoparticles do not agglomerate by like charges and not opposite charges between gelatin nanoparticles. However, when applying a crosslinking agent to crosslink the inside of gelatin nanoparticles so that the gelatin nanoparticles are not re-dissolved in water, it can be applied to a surfactant as a stabilizer to prevent the agglomeration between gelatin nanoparticles.

Examples of the crosslinking agent include a natural crosslinking agent such as genipin, glutaraldehyde or glyoxal. In order to separate the resulting gelatin nanoparticles from the used fatty acid and gelatin-dissolving solvent, it is subjected to centrifugation using a density difference between the gelatin nanoparticles and fatty acid and the gelatin-dissolving solvent. In order to remove fatty acid, the re-dispersion step of adding a polar or non-polar solvent may be performed once or repeated multiple times. Herein, the polar solvent includes solvents such as ethanol, methanol and ether, and the non-polar solvent includes solvents such as toluene, carbon tetrachloride, benzene and xylene. Subsequently, among the steps consisting of: freeze-drying of the re-dispersed gelatin nanoparticles, or dialysis or drying using a membrane, vacuum evaporation at a temperature of less than 40° C. that the gelatin nanoparticles are not re-dissolved, and separation and purification that the equivalent gelatin nanoparticles are not changed physico-chemically, one step may be performed or the same steps may be repeated or multiple steps comprising other step may be further performed to prepare gelatin nanoparticles.

Further, a mixture of hardly soluble drug-supported gelatin nanoparticles, fatty acids and gelatin dissolved solvents is not purified or separated, and the mixture is used without any change or emulsified and then applied to an oral formulation for the treatment of brain cancers or gastrointestinal cancers or a skin-cancer target as a transdermal absorption anticancer agents against skin cancer or the like.

Hereinafter, the method for preparing gelatin nanoparticles containing a hardly-water soluble drug will be more specifically described by way of examples.

### Example 1

### Preparation of Gelatin Nanoparticles in which Παχλιταξελ is Supported in the Inside of Nanoparticles

40 mg of gelatin was dissolved in 2 mL of DMSO while maintaining a temperature of 60° C. and stirred. 0.5 mg of paclitaxel was then added to the stirred solution. 1.5 mL of sorbitan sesquioleate as a surfactant was added to 30 mL of linoleic acid to prepare a solution. Then, the gelatin solution to which paclitaxel was added was added dropwise to linoleic acid solution. After 15 minutes, 96 uL of 5% glutaraldehyde solution as a crosslinking agent was added thereto. To cross-link the produced gelatin nanoparticles, the reaction mixture was stirred at 1000 rpm for about 12 hours. The solution containing the produced gelatin nanoparticles was centrifuged to 12000 g using a centrifuge for 15 minutes and then the linoleic acid supernatant was removed. The separated gelatin nanoparticles were added to 6 mL of ethanol and then vortex-dispersed. After performing the aforementioned centrifuring work, the step of removing supernatant was repeated two times. Then, to the separated gelatin nanoparticles, 3 mL of distilled water was added and re-dispersed. The reaction solution was pre-frozen to −75° C. and dried using a freeze dryer for 2 days. Meanwhile, the size and zeta potential of the gelatin nanoparticles were measured using a particle size measuring instrument (Malvern Co. zetarsize Nano ZS) for the solution containing the produced gelatin nanoparticles. As a result, it was confirmed that very uniform gelatin nanoparticles of about 200 nm was produced.

### Example 2

### Drug Release Experiment of Gelatin Nanoparticles in which Παχλιταξελ is Supported in the Inside of Nanoparticles

The drug release experiment was performed for the gelatin nanoparticles in which the paclitaxel prepared by using the preparation process of Example 1 was supported in the inside of nanoparticles. Three flasks were charged with 50 mL of PBS (pH 7.4), respectively, to which 10 mg of the produced paclitaxel-supported gelatin nanoparticles were added and then shaken at 100 rpm using a shaking incubator at 37° C. After shaking, 25 mg of trypsin was added to the respective flask after 24 hours. Each of the samples were taken, after shaking, on 10, 20 & 30 minutes, after 1, 2, 3, 4, 5, 6, 12 & 24 hours and one day, on 10, 20 & 30 minutes, 25, 26, 27, 30, 33, 45, 51, 69, 81, 94 & 100 hours, and the respective absorbances at a wavelength of 230 nm (paclitaxel) and 205 nm (linoleic acid+paclitaxel) were measured and then re-charged to each flask.

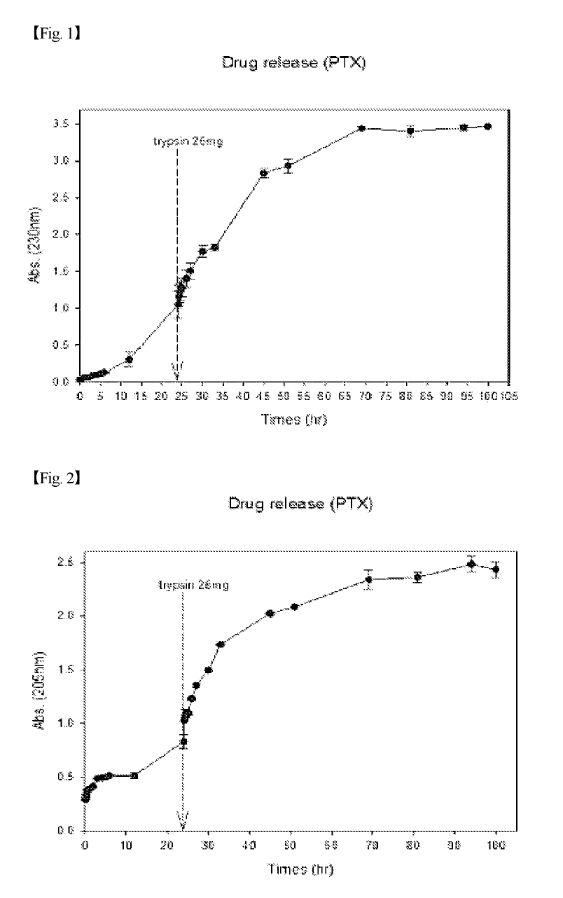

The absorbance transition at wavelength of 230 nm over time of the paclitaxel-supported gelatin nanoparticle samples from each flask is shown in FIG. 1. The absorbance up to 6 hours after shaking was slight, and after the lapse of 12 hours, it showed a little increase in the absorbance. However, from after 12 hours and until the lapse of 24 hours, it showed a remarkable increase trend in the absorbance. After addition of trypsin, while gelatin nanoparticles were decomposed, the absorbance increased rapidly, and the absorbance increase became gentle from 30 hours to 66 hours. Thereafter, the absorbance maintained a normal state up to 100 hours. Therefore, for 24 hours after shaking, 28.6% of the paclitaxel supported in the gelatin nanoparticles was released.

On the other hand, the encapsulation efficiency of gelatin nanoparticles of paclitaxel was 80.4% as a value of dividing the actual loading (0.1 mg PTX/10 mg NPs) by the theoretical loading (0.5 mg/(0.5 mg+40 mg). The amount of paclitaxel supported with gelatin nanoparticles and the amount of paclitaxel included in linoleic acid and ethanol supernatant which are discarded during the manufacture of the gelatin nanoparticles were 0.1 mg and 0.106 mg, respectively. Therefore, the remaining amount of paclitaxel not confirmed was 0.294 mg, corresponding to 58.8% (0.294 mg/0.5 mg) of the amount of paclitaxel initially injected in the gelatin solution. This was smaller than the centrifuged cut-off particle size and thus did not pelletize through centrifugation which was estimated as an amount of paclitaxel supported with gelatin nanoparticle discarded. In addition, the paclitaxel-support yield was 20% (0.1 mg/0.5 mg) which was lower than 37.5% (15 mg/40 mg) which was the yield of gelatin nanoparticles (Table 1).

TABLE 1
Results of the drug release experiment of gelatin
nanoparticles in which paclitaxel is supported in the inside
of nanoparticles.

| | | Unretrieved | | |
|---|---|---|---|---|
| | Retrieved | Gelatin NP | | |
| | Gelatin | (<cut-off | Discarded | |
| Component | nanoparticle (NP) | size) | Supernatant | Sum |
| Gelatin | 15 mg | 25 mg | 0 | 40 mg |
| | (37.5%) | (62.5%) | | (100%) |
| Paclitaxel | 0.1 mg | 0.294 mg | 0.106 mg | 0.5 mg |
| | (20%) | (58.8%) | (21.2%) | (100%) |

The cut-off size of the particles separated under centrifugation conditions such as a same angular velocity, a centrifugation time and density difference is proportional to the square root of the viscosity of the continuous phase fluid. The viscosity of the continuous phase linoleic acid was about 20.7 times than the viscosity of water at 20° C. and the cut-off size became 4.8 times larger.

Therefore, the yield of the gelatin nanoparticles became very smaller than when the continuous phase was water or ethanol.

Meanwhile, the absorbance transition over time at a wavelength of 205 nm is as shown in FIG. 2 and represents the sum of linoleic acid and paclitaxel. In FIG. 1 showing the transition of paclitaxel, a slight absorbance was shown up to 12 hours after shaking, while in FIG. 2 the rapid release was made for the same time, and the behavior of the normal state was shown from after 3-4 hours to 12 hours.

This shows that a small amount of linoleic acid absorbed on the outer surface of the gelatin nanoparticles was rapidly released in PBS. And the behavior after the adsorbed linoleic acid is depleted in 12 hours after shaking was consistent with the behavior of paclitaxel shown in FIG. 1. Therefore, while a small amount of linoleic acid adsorbed inside the gelatin nanoparticles inhibited the release of paclitaxel supported inside the gelatin nanoparticles, paclitaxel in the inside of the gelatin nanoparticles was released to the outside of the gelatin nanoparticles.

### INDUSTRIAL APPLICABILITY

According to the present invention, gelatin nanoparticles having a size of about 200 nm slowly releasing the hardly soluble drug-supported is used as a drug carrier. Thereby, the circulation time of the drug carrier within the human body is relatively prolonged as compared to a water-repellent drug. Also, EPR (Enhanced permeability and retention) effect for cancer cells is enhanced. Thus, the present invention is very useful for pharmaceutical and health functional food industry.

1. A method for preparing a drug delivery system characterized in that the water-repellent oil phase (O)/polar phase (W)/oil phase (O) emulsion system to which a surfactant was added to make a hardly-water soluble, water-repellent phase (O) drug into a fine particle which is soluble and easily absorbable, or the polar phase (W)/oil phase (O) emulsion system to which a surfactant is added without a hardly-water soluble drug, is produced without using a homogenizer, wherein a solvent of the polar phase (O) solution is diffused in the oil phase (O) and gelated while polar phase (W) droplets supported or not supported with the hardly-water soluble, water-repellent phase (O) drug are lowered to a nano-size in the O/W/O system or W/O system.

2. The method for preparing a drug delivery system according to claim 1 characterized in that the polar phase (W) drug is surrounded by the hydrophilic portion of the surfactant on the oil phase (O) to enhance the stability of the polar phase (W), and the hydrophilic side chain of the surrounded polar phase (W) is in contact with the hydrophilic portion of the surfactant.

3. The method for preparing a drug delivery system according to claim 1 characterized in that the hardly-water soluble, water-repellent phase (O) drug is in contact with the water-repellent side chain of the polar phase (W) in the inside of the polar phase (W) and thus the hardly-water soluble, water-repellent phase (O) drug is placed in the inside of the polar phase (W).

4. The method for preparing a drug delivery system according to claim 1 characterized in that the polar phase (W) drug is amphipathic.

5. A drug delivery system prepared by the method of claim 4.

6. The method for preparing a drug delivery system according to claim 1 characterized in that the water-repellent oil phase (O)/polar phase (W)/oil phase (O) emulsion system and the polar phase (W)/oil phase (O)/emulsion system are a hardly-water soluble, water-repellent drug phase/gelatin solution phase/fatty acid phase emulsion system or a gelatin solution phase/fatty acid phase emulsion system, the polar phase (W) droplet supported or not supported with the hardly-water soluble, water-repellent phase (O) drug is a gelatin droplet supported or not supported with the hardly-water soluble, water-repellent phase (O) drug, and the particle gelated while the polar phase (W) droplet becomes a nano-size is a gelatin particle.

7. The method for preparing a drug delivery system according to claim 1 characterized in that the hardly-water soluble, water-repellent phase drug is one or more selected from the group consisting of paclitaxel, coenzyme Q10, ursodeoxycholic acid, ilaprazole and imatinib mesylate.

8. The method for preparing a drug delivery system according to claim 6 characterized in that the hardly-water soluble, water-repellent drug phase/gelatin solution phase/fatty acid phase emulsion system is constructed by adding a hardly soluble drug to a gelatin in which gelatin is dissolved at 40-60° C. using the polar phase (W) solvent; adding dropwise or continuously adding the gelatin solution to fatty acid.

9. The method for preparing a drug delivery system according to claim 6 characterized in that the gelatin solution phase/fatty acid phase emulsion system is constructed by adding dropwise or continuously adding to fatty acid a gelatin solution in which gelatin is dissolved at 40-60° C. using the polar phase (W) solvent.

10. The method for preparing a drug delivery system according to claim 1 characterized in that the polar phase (W) solvent is DMSO.

11. The method for preparing a gelatin nanoparticle according to claim 8 characterized in that the fatty acid is an unsaturated fatty acid.

12. The method for preparing a gelatin nanoparticle according to claim 8 characterized in that the fatty acid is a liquid phase at room temperature.

13. The method for preparing a gelatin nanoparticle according to claim 11 characterized in that the unsaturated fatty acid is oleic acid or linoleic acid.

14. The method for preparing a gelatin nanoparticle system according to claim 8 characterized in that a surfactant is added to the fatty acid.

15. A gelatin nanoparticle prepared by the method of claim 14.

16. The method for preparing a drug delivery system according to claim 1 characterized in that the surfactant is added in an amount of 1 to 2 w/v % with respect to the volume of fatty acid or 30 to 35 times of the total weight of the gelatin to fatty acid prior to the production of the hardly-water soluble, water-repellent drug phase/gelatin solution phase/fatty acid phase emulsion system or the gelatin solution phase/fatty acid phase emulsion system, or the surfactant is added to fatty acid within one hour after the production of the hardly-water soluble, water-repellent drug phase/gelatin solution phase/fatty acid phase emulsion system or the gelatin solution phase/fatty acid phase emulsion system.

17. The method for preparing a drug delivery system according to claim 1 characterized in that the surfactant is one or more sorbitan-based surfactants selected from the group consisting of sorbitan monoisostearate, sorbitan monooleate, sorbitan sesquioleate and sorbitan trioleate.

18. The method for preparing a drug delivery system according to claim 6 characterized in that a crosslinking agent is injected in an amount of 100 to 200 ug per mg of the entire gelatin after the production of the hardly-water soluble, water-repellent drug phase/gelatin solution phase/fatty acid phase emulsion system or the gelatin solution phase/fatty acid phase emulsion system, and then stirred for 10 to 15 hours to perform the crosslinking reaction.

19. The method for preparing a drug delivery system according to claim 18 characterized in that the crosslinking agent includes ginipin, glutaraldehyde or glyoxal.

20. The method for preparing a drug delivery system according to claim 6 characterized in that the gelatin gel particles supporting the hardly-water soluble, water-repellent phase (O) drug is obtained by centrifuging the hardly-water soluble, water-repellent phase drug phase/gelatin gel particle phase/fatty acid phase suspension system; performing a re-dispersion step of adding a solvent once or repeating it multiple times; subsequently performing one step among the steps of dialysis or drying using a membrane, vacuum evaporation, separation and purification that the equivalent gelatin nanoparticles are not changed physico-chemically, repeating the same steps or performing multiple steps comprising other step.

21. The method for preparing a drug delivery system according to claim 6 characterized in that the gelatin gel particles not supporting the hardly-water soluble, water-repellent phase (O) is obtained by centrifuging the gel particle phase/fatty acid phase suspension system; performing a re-dispersion step of adding a solvent once or repeating it multiple times; subsequently, performing one step selected among dialysis or drying using a membrane, vacuum evaporation, separation and purification that the equivalent gelatin nanoparticles are not changed physico-chemically, or repeating the same steps or additionally performing multiple steps comprising other step.

22. The method for preparing a drug delivery system according to claim 8 characterized in that the gelatin concentration of the gelatin solution is 0.01 g/mL to 0.03 g/mL.

23. The method for preparing a drug delivery system according to claim 6 characterized in that the volume of the fatty acid phase is 15 to 20 times of the total volume of the polar phase (W) solvent to be added.

24. The method for preparing a drug delivery system according to claim 20 characterized in that the average size of the gelatin gel particle is 200 nm.

25. A drug delivery system prepared by the method of claim 24.
